(19) United States
(12) Patent Application Publication
(10) Pub. No.: US 2006/0034940 A1
(43) Pub. Date: Feb. 16, 2006

(54) Drinking water container containing gemstones and crystals

(76) Inventor: Lynda Lee Bell, Wilmette, IL (US)

Correspondence Address: MCANDREWS HELD & MALLOY, LTD, 500 WEST MADISON STREET, SUITE 3400, CHICAGO, IL, 60661, US

(21) Appl. No.: 11/194,825

(22) Filed: Aug. 1, 2005

Related U.S. Application Data
(63) Continuation of application No. 10/794,284, filed on Mar. 8, 2004, now abandoned.
(63) Continuation of application No. 10/790,284, filed on Mar. 1, 2004, now abandoned.

(51) Int. Cl. B65D 1/02; A61K 33/00

(52) U.S. Cl. 424/600; 215/40

(57) ABSTRACT

A bottle of drinking water includes a transparent body having a top with a neck portion to provide an outlet and a bottom upon which the body rests. A plurality of gemstones are arranged in the water within the body. The gemstones may move within the water as the body is moved during consumption of the water.

### BACKGROUND OF THE INVENTION

1. Field of the Invention

This invention relates in general to drinking water bottles and more particularly, to drinking water bottles containing gemstones or crystals.

2. Summary of the Prior Art

Stones in the form of precious and semi-precious gemstones, and crystals have long been said to provide unique benefits to human health and well being. Among many gemstones, agate, amethyst, aventurine, carnelian, citrine, jasper, and sodalite demonstrate special properties for health, well being and energy. The foregoing beneficial effects have been observed in the past when drinking water charged with such gemstones or crystals, is consumed.

One theory relating to the benefits of quartz and gemstones charged drinking water is based on areas of the body, which are known as chakras, based on age old Tibertian beliefs. Chakras are defined as main energy centers of the body which are visualized as like spirals of energy. Chakra is derived from the Sanskrit term for “wheel”. Chakras are thought to be energy centers in which a balance of these energies benefit health and/or provide a sense of well being. Each chakra is associated with a particular semi-precious stone. Drinking water charged with such semi-precious stones and crystals act to balance the energy associated with a chakra. Moreover, the presence of gemstones and crystals in drinking water have also been observed as being highly beneficial without considering the theory relating to chakras.

### SUMMARY OF THE INVENTION

It is therefore an objective of the invention to provide several embodiments of a water bottle having gemstones or crystals immersed in the water. Consumption of the water from the bottle of the invention provides health benefits and a feeling of well being among numerous other improved physical and mental attributes.

### BRIEF DESCRIPTION OF THE DRAWINGS

FIG. 1 is a front elevational view of a first embodiment of the water bottle containing gemstones or crystals of the invention;

FIG. 2 is a front elevational view of the second embodiment of the water bottle containing gemstones or crystals of the invention;

FIG. 3 is a front elevational view of the third embodiment of the water bottle containing crystals of the invention.

FIG. 4 is a front elevational view of the fourth embodiment of the water bottle containing gemstones or crystals of the invention; and

FIG. 5 is a front elevational view of the fifth embodiment of the water bottle containing gemstones or crystals of the invention.

### DESCRIPTION OF THE PREFERRED EMBODIMENTS

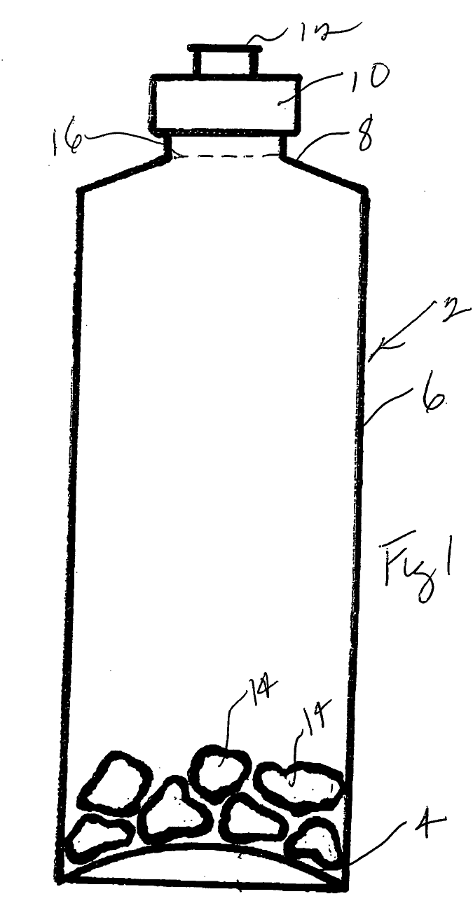

Referring to FIG. 1, there is illustrated a first embodiment of the water bottle of the invention containing crystals or semi-precious gemstones, generally designated by reference numeral 2. Water bottle 2 includes a closed bottom 4 and a hollow bottle 6 having a square cross-section that tapers into a neck portion 8. A standard bottle cap 10 having a nozzle portion 12 is attached to the neck 8 in a conventional manner. Although the body 6 is described as having a square cross-section, it may have other cross sectional shapes (not shown), such as a circular and the like. The water bottle 2 is preferably transparent and molded or otherwise formed from a plastic or glass material.

The container is filled with drinking water, such as purified water, spring water, or other popular water forms, for consumption and contains a plurality of gemstones or crystals 14 loosely arranged at the bottom of bottle 6 in an upright position of the bottle to alter the properties of the water. The neck portion 8 includes a perforated filter 16 to permit the passage of water for consumption or pouring, but further acts as a retention element to prevent the passage of gemstones or crystals from the bottle. Although not intended to be so limited, gemstones to be placed in the bottle may be agate (blue lace), amethyst (purple), aventurine (green), carnelian (orange), citrine (yellow), jasper (red), or sodalite (navy). Other semi-precious or precious gemstones, or crystals may be used that demonstrate the desired properties when placed in water in accordance with the invention.

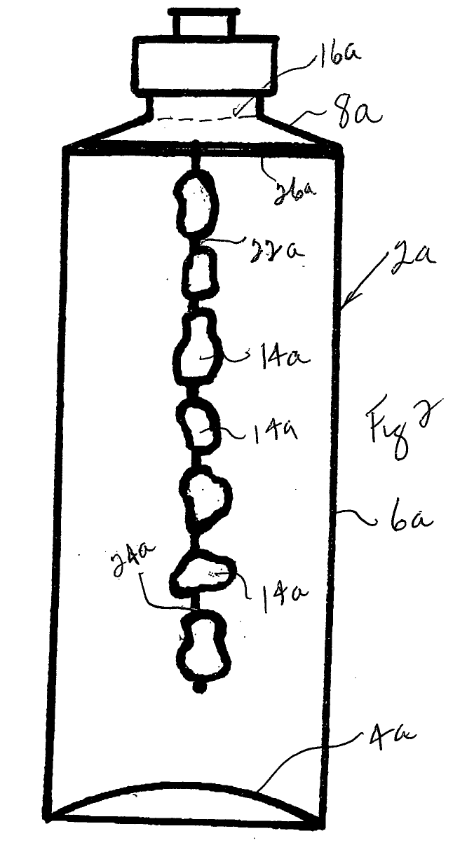

Referring to FIG. 2, there is illustrated the second embodiment of the invention, generally designated by reference numeral 2a. In the embodiment of FIG. 2, the water bottle 6a, neck 8a and cap 10a are identical to the corresponding elements described with reference to FIG. 1. In FIG. 2, the plurality of gemstones or crystals 14a are retained in the water bottle 6a on a suspended line 22a, formed from nylon or the like. The gemstones or crystals 14a have holes 24a through which the line 22a extend. The line 22a includes an enlarged end 28 to retain the gemstones or crystals on the line. The gemstones or crystals 14a may contact each other along the line 22a or be separated from each other along the line 22a by a suitable technique. The upper end of the line 22a may be suspended at its upper end or on a horizontal rod 26a and the like suitably affixed within the bottle 6a. Other means of suspension of the line 22a may be employed, if desired.

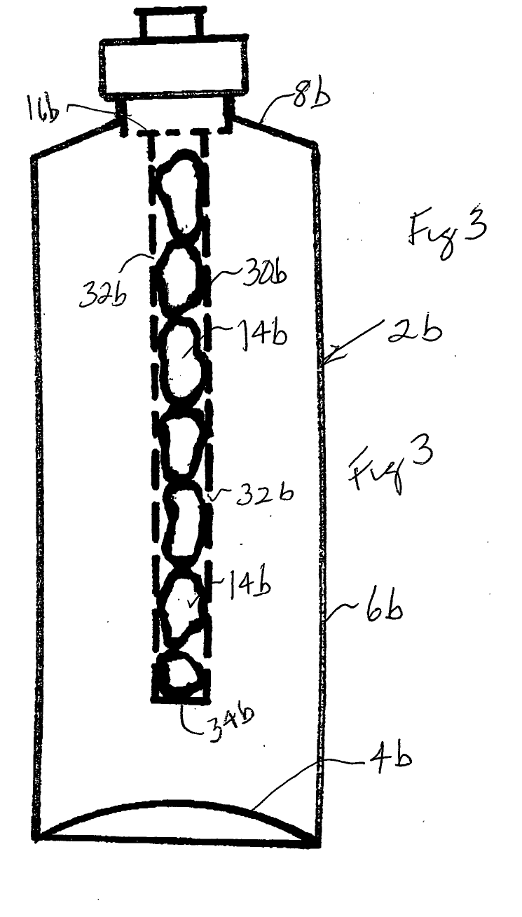

Referring to FIG. 3, there is illustrated a third embodiment of the invention, generally designated by reference numeral 2b. The embodiment of FIG. 3 includes a bottle body 6b, neck 8b, and cap 10b of similar design as the bottle construction described with reference to FIG. 1. The gemstones or crystals 14b of a type as previously described are retained in bottle 6b within a vertical clear hollow tube 30b. The tube 30b is cylindrical in shape and is formed from a clear plastic. The tube 30b has a plurality of apertures 32b to provide contact between the water and gemstones or crystals 14b. The apertures 32b are smaller than the smallest of the gemstones or crystals to prevent passage from the tube 30b. The bottom 34b of tube 30b is closed to further retain the gemstones or crystals. A perforated filter 36b is attached to the neck 8b to prevent escape of the tube 30b or gemstones or crystals from the bottle. The tube 30b may be suspended in a suitable manner on the filter 16. The tube 30b may be in the form of a modified drinking straw or be a molded or extruded plastic hollow member.

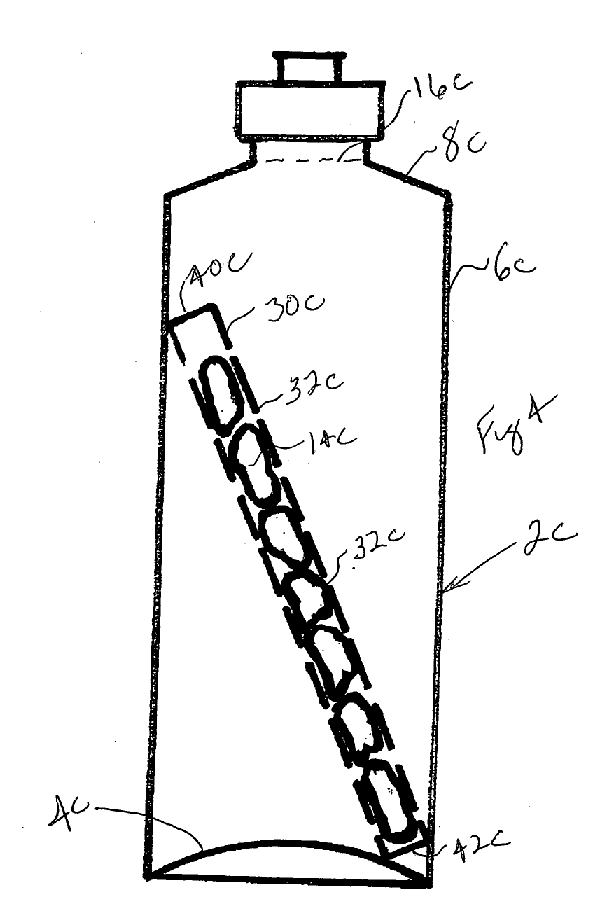

Referring to FIG. 4, there is illustrated a fourth embodiment of the invention, generally designated by reference numeral 2c. The water bottle 2c of the invention is similar to the embodiment of FIG. 3, except that tube 30c is not retained within the bottle body 6c, but is unretained. The ends of tube 30c are closed by end walls 40c, 42c. The perforations 32 are smaller than the gemstones or crystals 14c to prevent egress from the tube 30c.

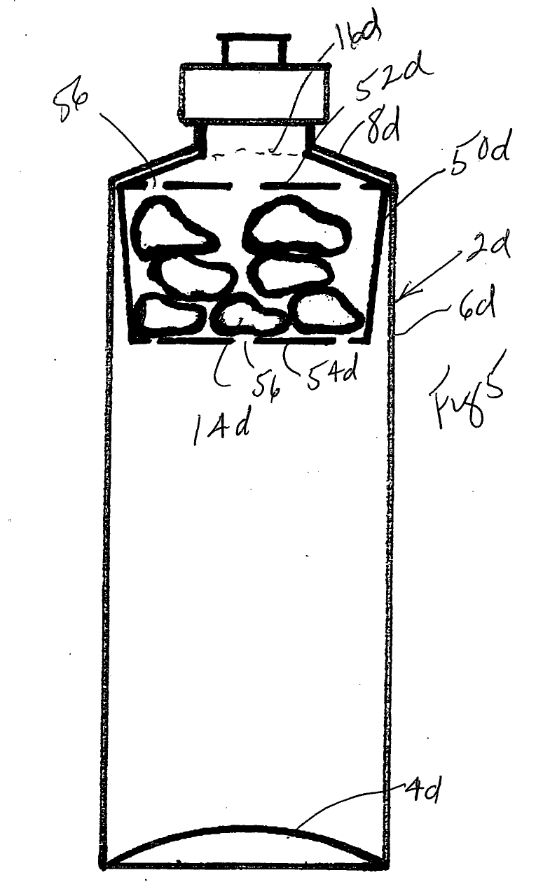

Referring now to FIG. 5, there is illustrated a fifth embodiment of the invention, generally designated by reference numeral 2d. In FIG. 5, the bottle body 6d, neck 8d, and cap 10d are similar to what was described with reference to preceding embodiments. A plastic basket 50d is suspended at the top of the interior of body 6d by a suitable technique. The plastic basket 50d retains the gemstones and crystals within the bottle 6d. The basket 50d may have a square, or other circular cross-sectional shape as is desired. A filter 16d is provided at the neck portion 8d to ensure the prevention passage of gemstones or crystals in the event they accidentally escaped from basket 50d. The top and bottom walls of basket 50d, 52d, 54d have perforations 56 to allow the water to pass through the basket 50d in contact with the gemstones or crystals contained therein.

1. A bottle of drinking water, of a size for allowing consumption directly from the bottle, comprising:
a transparent body forming a hollow bottle having a predetermined size, said body having a top, and a bottom upon which said body rests;
drinking water contained within said body;
a hollow neck portion integrally formed at the top of said body and creating an outlet to permit passage of said water for consumption;
a plurality of gemstones disposed in a linear array within said transparent body and in contact with said water,
a transparent tube holding each of said gemstones and retaining said gemstones in said linear array; said transparent tube being unrestrained within said body and being movable within said water as said body is moved during said consumption of said water.

2. (canceled)

3. The water bottle according to claim 1 wherein said transparent tube has a plurality of apertures to permit water to contact said plurality of said gemstones, said apertures being smaller than said plurality of gemstones to prevent the passage of said gemstones through said apertures.

4. The water bottle according to claim 3 wherein said transparent tube is cylindrical in shape.

5-9. (canceled)

10. A water bottle comprising
body means forming a hollow bottle having a predetermined width, and receiving drinking water, a hollow neck portion integrally formed on said bottle creating an outlet flow passage,
a plurality of crystals of a predetermined size being submerged in the water in said bottle, and;
retention means permitting the passage of water and retaining said crystals within said bottle.

11. The water bottle according to claim 10 wherein said retention means is a perforated filter mounted across said flow passage.

12. The water bottle according to claim 10 wherein said retention means is a hollow bottle containing said plurality of said crystals, having perforations to permit water to contact said plurality of said crystals, said perforations being smaller than said predetermined size of said plurality of crystals to prevent the passage o said crystals through said perforations.

13. The water bottle according to claim 12 wherein said bottle is an elongated tube having said plurality o perforations.

14. The water bottle according to claim 13 further including means for suspending said hollow tube from an upper portion of said bottle.

15. The water bottle according to claim 12 wherein said retention means is a hollow basket containing said plurality of crystals.

16. The water bottle according to claim 15 wherein said basket has a width substantially equal to said predetermined width of said bottle.

17. The water bottle according to claim 15 further includes means for suspending said basket at an upper portion of said bottle.

18. (canceled)
